(19) United States
(12) Patent Application Publication
(10) Pub. No.: US 2008/0075664 A1
(43) Pub. Date: Mar. 27, 2008

(54) Control Of Diabetes And Obesity

(75) Inventor: Amy S. Lee, San Marino, CA (US)

Correspondence Address: FISH & RICHARDSON P.C., P.O BOX 1022, Minneapolis, MN, 55440-1022, US

(73) Assignee: UNIVERSITY OF SOUTHERN CALIFORNIA, Los Angeles, CA (US)

(21) Appl. No.: 11/844,721

(22) Filed: Aug. 24, 2007

Related U.S. Application Data
(60) Provisional application No. 60/824,415, filed on Sep. 1, 2006.

(51) Int. Cl. A61K 31/353; A01K 67/00; A61K 31/6615; A61K 39/395; A61P 3/04; A61P 3/10; G01N 33/15

(52) U.S. Cl. 424/9.2; 424/130.1; 514/102; 514/456; 800/9

(57) ABSTRACT

Provided are methods of increasing resistance to and methods of treating disorders associated with weight gain. Also provided are methods of screening for an agent that prevents or treats a disorder associated with weight gain using animal models heterozygous for IP3R1.

### CROSS REFERENCE TO RELATED APPLICATIONS

The present application claims priority to U.S. Ser. No. 60/824,415 filed Sep. 1, 2006, which is incorporated by reference herein in its entirety.

### STATEMENT REGARDING FEDERALLY FUNDED RESEARCH

This invention was made with support from Grant No. 53-5114-5500 from the National Institutes of Health and from Grant No. 53-5114-7320 from the National Institute of Diabetes and Digestive and Kidney Diseases. The U.S. government may have certain rights in this invention.

### BACKGROUND

The endoplasmic reticulum (ER) is a perinuclear, cytoplasmic compartment where proteins and lipids are synthesized. It is also a major intracellular storage compartment for Ca2+, which plays a central role in cellular signaling. Most secreted and integral membrane proteins of mammalian cells are translocated cotranslationally into the lumen of the ER, which provides a specialized environment for the posttranslational modification and folding of secreted, transmembrane and resident proteins of the various compartments of the endomembrane system. Proteins that are properly folded and assembled into oligomeric structures as needed will be cleared for exit from the ER, whereas unfolded/malfolded proteins will either be retained in the ER through interaction of molecular chaperones localized in the ER, or they would be disposed of by an ER-associated protein degradation machinery (Ellgaard et al., 1999).

ER stress can be triggered by an increase in synthesis of proteins such that it exceeds the capacity of the folding machinery, and it also occurs under pathophysiological conditions such as hypoglycemia which could lead to the production and accumulation of underglycosylated proteins in the ER (Kaufman et al., 2002). Proper function of the ER is essential to cellular homeostasis and cell survival, especially for specialized secretory cells such as the insulin-producing β-cell, which processes various and large amounts of secretory proteins. A major contributing factor to diabetes is impaired insulin signaling and decreased insulin secretion. Pancreatic β-cells are the only cell that synthesizes and secretes insulin, and therefore it plays a critical role in glucose homeostasis. Unlike type I diabetes which is caused by excessive loss of β-cells due to autoimmune activity, dysfunction of β-cells is common feature of type II diabetes (Mathis et al., 2001). In type II diabetes, hyperglycemia can develop when β-cells fail to compensate for the increased demand for insulin secretion. However, persistent high levels of insulin will result in insulin resistance and subsequent development of obesity and diabetes.

Unlike other cell types, production of glucose 6-phosphate and metabolic flux through glycolysis in β-cells is controlled by glucokinase rather than the low Kₘ-hexokinases (Kaufman et al., 2002). Consequently, β-cells are unique in that their cellular [ATP/ADP] ratio decreases to low levels when blood glucose is basal (5 mM). Assuming that periodic decreases in the [ATP/ADP] could compromise protein folding in the ER, the ER compartment of the β-cell could be more exposed to energy or redox fluctuations than in other cell types when glucose levels vary within the normal physiological range (3 to 10 mM), as occurs between meals. In that case, β-cells would uniquely require specific cellular defensive mechanisms against ER stress for survival. In support, ER stress in β-cells could cause apoptosis and lead to the development of diabetes. One example is the Akita mouse which is characterized by hyperglycemia with reduced β-cell mass without insulitis or obesity (Kayo and Koizumi, 1998). The Akita mouse suffers from a mutation resulting in the disruption of a disulphide bond formation between the A and B chain of proinsulin, inducing a conformational change. In another example, mutations affecting the ER stress-activated pancreatic ER kinase and its downstream effector, the translation initiation complex eukaryotic initiation factor, negatively impact islet cell development, function and survival and are associated with the Wolcott-Rallision syndrome of infantile diabetes (Harding et al., 2001). Collectively, these findings suggest that insulin-secreting p-cell is particularly sensitive to the adverse effects of perturbed ER function and may have adapted specific mechanisms towards its protection and survival. One such mechanism is the activation of ER stress protective genes.

Inbred mouse genetic models bearing specific gene mutations have provided important insight into the pathogenesis of obesity and/or diabetes (Allayee et al., 2006). Among the best studied and well-established models are the insulin receptor pathway knockout models (Rhodes and White, 2002), the obese leptin-deficient ob/ob mouse (Pelleymounter et al., 1995), mouse bearing mutations in the leptin receptor (Chua et al., 1996), the yellow coat color agouti (Avy/a) mouse with a mutation in the agouti gene that causes ubiquitous expression of the encoded protein (Duhl et al., 1994). By crossing with the agouti mice, obese mice overexpressing the human islet amyloid polypeptide have been generated (Butler et al., 2003). Despite these advances, each model also has specific limitations. For example, some of these mutations are very rare among human populations and they may also affect other peripheral functions and pathways, giving rise to unknown side effects unrelated to diabetes.

### SUMMARY

Provided are methods of increasing resistance to and methods of treating disorders associated with weight gain. The methods contain the steps of selecting a subject at risk for developing or having a disorder associated with weight gain and administering to the subject one or more agents that inhibit expression or activity of GRP78 or IP3R1. Also provided are two non-human animal models heterozygous for Grp78 or IP3R1. Provided are methods of screening for an agent that prevents or treats a disorder associated with weight gain using the animal model heterozygous for IP3R1.

The details of one or more embodiments are set forth in the accompanying drawings and the description below. Other features, objects, and advantages will be apparent from the description and drawings, and from the claims.

### DESCRIPTION OF DRAWINGS

FIG. 1 is a schematic showing mechanisms for the Grp78+/− and Opt mouse model phenotypes.

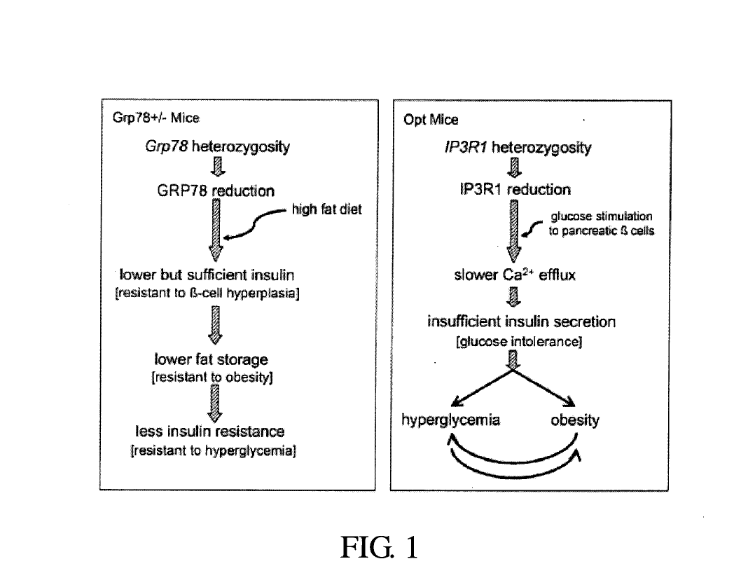

### DETAILED DESCRIPTION

The ER is a cellular organelle where secretory and membrane proteins are synthesized and modified and is also a major intracellular Ca²⁺ storage compartment. The glucose regulated protein GRP78, also referred to as the immunoglobulin binding protein, BiP, is a central regulator for ER function due to its role in protein folding and assembly, targeting misfolded protein for degradation, ER Ca²⁺ binding and controlling the activation of trans-membrane ER stress sensors. The activation of the gene encoding GRP78 (Grp78) is widely used as a monitor for ER stress and has led to the discoveries of several unique signaling pathways whereby stress in this critical organelle is transmitted to the nucleus to initiate the unfolded protein response (UPR), which triggers multiple pathways to allow cells to respond to stress conditions that target the ER. As a major ER chaperone protein and a master regulator of ER signaling, GRP78 is predicted to be particularly critical for highly secretory cells such as pancreatic β-cells, which has an active ER devoted to insulin biosynthesis and is specifically sensitive to physiological fluctuations in blood glucose. Recently, obesity has been linked to ER stress; however, the basic mechanisms are just emerging. In the obese leptin deficient ob/ob mouse, GRP78 expression is spontaneously induced; however, its functional significance is not known. In mice subjected to high fat diet, GRP78 level is also elevated, indicative of ER stress. Towards understanding the regulation and function of GRP78 in vivo, targeted mutation of the Grp78 allele in a mouse model was generated (Luo et al., 2006). Transgenic mouse lines where the Grp78 promoter drives expression of the β-galactosidase (β-gal) reporter gene were also developed (Mao et al., 2004).

GRP78 was first discovered as a 78,000 dalton protein whose synthesis was enhanced in tissue culture cells grown in medium deprived of glucose (Shiu et al., 1977). Subsequently, GRP78 was determined to be an ER resident protein and its synthesis can be stimulated by a variety of environmental and physiological stress conditions that perturb ER function and homeostasis (Lee, 1987; 2001). GRP78 is also commonly referred to as BiP, the immunoglobulin heavy chain binding protein. BiP was originally found to bind to the immunoglobulin heavy chains of pre-B cells (Haas and Wabl, 1983). Through analysis of proteins related to the heat shock protein family (HSP70) and using an antibody against BiP, it was discovered that BiP is identical to the previously reported GRP78; furthermore, it was determined that BiP is not restricted to B cells. In fact, the level of BiP in pre-B and B cells is comparable to that found in fibroblasts and its level is greatly elevated in antibody secreting plasma cells (Munro and Pelham, 1986).

A large amount of work has established that specific induction of GRP78 is indicative of ER stress. ER stress can occur under various physiological settings that have significant implications in health and disease (Lee, 2001; Rutkowski and Kaufman, 2004). For example, in highly specialized secretory cells such as plasma cells and pancreatic β-cells, the ER compartment is expanded considerably and because of the high volume of protein traffic, the ER can experience accumulation of partially folded proteins that require chaperone assistance. Malfolded protein accumulation has also been associated with neurodegenerative disorders such as Alzheimer's and Parkinson's diseases, as well as prion protein diseases. Further, induction of GRP78 in multiple types of solid tumors can be attributed to the much higher glucose utilization rates of cancer cells, compounded by glucose and oxygen starvation resulting from poor perfusion within tumors (Gazit et al., 1999; Dong et al., 2004).

In addition to being an essential and major chaperone protein, GRP78 binds Ca²⁺ and serves as an ER stress signaling regulator (Kaufman, 1999; Reddy et al., 2003). It is well accepted that GRP78 is a key rheostat in controlling ER homeostasis. In mammalian cells, several ER-resident transmembrane proteins have been identified that act as transducers of ER stress signaling: the serine/threonine kinase and endoribonuclease IRE1, the PERK serine/threonine kinase (also referred to as PEK) and the basic leucine-zipper transcription factor ATF6 (Rutkowski and Kaufman, 2004; Sommer and Jarosch, 2002). In non-stressed cells, GRP78 binds to all three transducers which are maintained in an inactive state. Upon ER stress, all three sensors are released from GRP78. In the case of IRE1 and PERK, they homodimerize through their luminal domains, autophosphorylate their respective cytoplasmic domains, and become activated (Bertolotti et al., 2000). For ATF6, a fraction of it is translocated from the ER to the Golgi complex, where it is cleaved by the proteases S1P and S2P. The cleaved form of ATF6 enters the nucleus and acts as an active transcription factor for the UPR target genes, including Grp78 (Ye et al., 2000; Shen et al., 2002; Hong et al., 2004). Therefore, GRP78 is a key regulator of ER stress transducers since their activation upon ER stress is dependent on their release from GRP78. Recently, two independent groups have detected that a fraction of GRP78 can exist as a transmembrane ER protein (Reddy et al., 2003; Rao et al., 2002). This implies that GRP78 can potentially interact directly with the cytosolic components. For example, GRP78 has been reported to form complexes with procaspases such as caspase-7 and mouse caspase-12 that associate with the outer ER membrane. Through these interactions, either directly or indirectly, GRP78 regulates the balance between cell survival and apoptosis in ER-stressed cells.

As described in the Examples below, heterozygous Grp78+/− mice expressing half of wild-type (WT) GRP78 protein levels were resistant to high fat diet (HFD) induced obesity and diabetes despite normal food intake and lipid absorption.

IP3R1 (inositol 1,4,5-triphosphate receptor 1) is an ER transmembrane protein and is predominant in mouse brain and pancreatic islets as the major regulator of Ca²⁺ efflux from the ER (Lee and Laychock, 2001). The IP3R1 protein binds the intracellular second messenger IP3, which is generated by phsopholipase C-mediated hydrolysis of phosphatidylinositol 4,5-bisphosphate (Berridge, 1993). Ligand binding triggers the efflux of calcium from intracellular stores, suggesting the IP3R1 is both a receptor of IP3 and a Ca²⁺ channel. Described herein is a mouse model, referred to as D2D, containing a transgene inserted into mouse chromosome 6, disrupting the Ca²⁺ receptor gene IP3R1. In the D2D mouse model, the Grp78/p-gal transgene was found to be inserted into the middle of the gene encoding the inositol 1,4,5-triphosphate receptor type 1 (IP3R1), which is a major ER Ca²⁺ receptor controlling ER Ca²⁺ efflux in mouse pancreatic β-cells. The IP3R1 protein in D2D mice was half of WT level. D2D mice, fed on regular diet, develop age-dependent onset of mild obesity, glucose intolerance and hyperglycemia. Further analysis revealed that in both models, fasting insulin level is lower in the mutant mice compared to the WT siblings under identical diet conditions.

Thus, provided are methods of increasing resistance to a disorder associated with weight gain in a subject comprising selecting a subject at risk for developing a disorder associated with weight gain and administering to the subject one or more agents that inhibit expression or activity of GRP78. Also provided are methods of treating a disorder associated with weight gain in a subject comprising selecting a subject that has a disorder associated with weight gain and administering to the subject one or more agents that inhibit expression or activity of GRP78. The methods can also comprise administering a second therapeutic agent to the subject. The disorder associated with weight gain can be, for example, obesity or type II diabetes. The obesity can be diet-induced obesity. The phrase weight gain refers to the status of a subject as overweight or obese based on generally accepted medical standards.

Provided are also methods of increasing resistance to a disorder associated with weight gain in a subject comprising selecting a subject at risk for developing a disorder associated with weight gain and administering to the subject one or more IP3R1 agonists. Provided are methods of treating a disorder associated with weight gain in a subject comprising selecting a subject that has a disorder associated with weight gain and administering to the subject one or more IP3R1 agonists. The methods can also comprise administering a second therapeutic agent to the subject. The disorder associated with weight gain can be, for example, obesity or type II diabetes. The obesity can be diet-induced obesity.

The provided methods comprise administering an agent that reduces or inhibits expression or activity of Grp78. Reduction or inhibition of Grp78 can comprising inhibiting or reducing expression of Grp78 mRNA or Grp78 protein, such as by administering antisense molecules, triple helix molecules, ribozymes and/or siRNA. grp78 gene expression can also be reduced by inactivating the grp78 gene or its promoter. The nucleic acids, ribozymes, siRNAs and triple helix molecules for use in the provided methods may be prepared by any method known in the art for synthesis of DNA and RNA molecules. These include techniques for chemically synthesizing oligodeoxyribonucleotides and oligoribonucleotides well known in the art such as for example solid phase phosphoramide chemical synthesis. Alternatively, RNA molecules may be generated by in vitro and in vivo transcription of DNA sequences encoding the nucleic acid molecule. Such DNA sequences may be incorporated into a wide variety of vectors, which incorporate suitable RNA polymerase promoters. Antisense cDNA constructs that synthesize antisense RNA constitutively or inducibly, depending on the promoter used, can be introduced stably into cell lines.

In addition, reduction or inhibition of Grp78 includes inhibiting the activity of the Grp78 protein using agents referred to herein as Grp78 antagonists. Drugs which target Grp78 have been developed (Ermakova et al., Cancer Res. 66:9260-9 (2006); Davidson et al., Cancer Res. 65:4663-72 (2005); Zhou et al., J. Natl. Cancer Inst. 90:381-88 (1998); Arap et al., Cancer Cell 6:275-84 (2004); Park et al., J. Natl. Cancer Inst. 96:1300-10). Grp78 antagonists also include antibodies, soluble domains of Grp78 and polypeptides that interact with Grp78 to prevent Grp78 activity. The nucleic acid and amino acid sequence of Grp78 is known in the art. Therefore, variants and fragments of Grp78 that act as Grp78 antagonists can be prepared by any method known to those of skill in the art using routine molecular biology techniques. Typical agents for inhibiting or reducing (e.g., antagonistic) activity of GRPs include mutant/variant GRP polypeptides or fragments and small organic or inorganic molecules.

Inhibitors of Grp78 include inhibitory peptides or polypeptides. As used herein, the term peptide, polypeptide, protein or peptide portion is used broadly herein to mean two or more amino acids linked by a peptide bond. Protein, peptide and polypeptide are also used herein interchangeably to refer to amino acid sequences. The term fragment is used herein to refer to a portion of a full-length polypeptide or protein. It should be recognized that the term polypeptide is not used herein to suggest a particular size or number of amino acids comprising the molecule and that a peptide of the invention can contain up to several amino acid residues or more. Inhibitory peptides include chimeric peptides with Grp78 binding motifs fused to pro-apoptotic sequences (Arap et al., Cancer Cell 6:275-84 (2004), which is incorporated by reference herein in its entirety). Inhibitory proteins also include Kringle 5 (K5), melanoma differentiation-associated gene-7/interleukin-24 (MDA7/IL-24) and activated form of α-2 macroglobulin (Davidson et al., Cancer Res. 65:4663-72 (2005); Dent et al., J. Cell Biochem. 95:712-9 (2005); Misra et al., J. Biol. Chem. 281:3694-707 (2006), which are incorporated by reference herein in their entireties).

Inhibitory peptides include dominant negative mutants of a Grp78. Dominant negative mutations (also called antimorphic mutations) have an altered phenotype that acts antagonistically to the wild-type or normal protein. Thus, dominant negative mutants of Grp78 act to inhibit the normal Grp78 protein. Such mutants can be generated, for example, by site directed mutagenesis or random mutagenesis. Proteins with a dominant negative phenotype can be screened for using methods known to those of skill in the art, for example, by phage display.

Proteins that inhibit Grp78 include antibodies with antagonistic or inhibitory properties. Antibodies to Grp78 are commercially available, for example, from Santa Cruz Biotechnology (Santa Cruz, Calif.). In addition to intact immunoglobulin molecules, fragments, chimeras, or polymers of immunoglobulin molecules are also useful in the methods taught herein, as long as they are chosen for their ability to inhibit Grp78. The antibodies can be tested for their desired activity using in vitro assays, or by analogous methods, after which their in vivo therapeutic or prophylactic activities are tested according to known clinical testing methods.

Nucleic acids that encode the aforementioned peptide sequences are also disclosed. These sequences include all degenerate sequences related to a specific protein sequence, i.e., all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. A wide variety of expression systems may be used to produce peptides as well as fragments, isoforms, and variants. Such peptides or proteins are selected based on their ability to reduce or inhibit expression or activity of Grp78.

Inhibitors of a Grp78 also include, but are not limited to, genistein, (−)-epigallocatechin gallate (EGCG), salicyclic acid from plants, bacterial AB₅ subtilase cytoxin, versipelostatin (Ermakova et al., Cancer Res. 66:9260-9 (2006); Zhou and Lee, J. natl. Cancer Inst. 90:381-8(1998); Deng et al., FASEB J. 15:2463-70(2001); Montecucco and Molinari, Nature 443:511-2 (2006); Park et al., J. Natl. Cancer Inst. 96:1300-10 (2004), which are incorporated herein in their entireties). Inhibitors of GRP78 also include taxanes, such as, for example, paclitaxel and docetaxel in combination with doxirubicin.

Also provided herein are functional nucleic acids that inhibit expression of Grp78. Such functional nucleic acids include but are not limited to antisense molecules, aptamers, ribozymes, triplex forming molecules, RNA interference (RNAi), and external guide sequences. Thus, for example, a small interfering RNA (siRNA) could be used to reduce or eliminate expression of Grp78.

Functional nucleic acids are nucleic acid molecules that have a specific function, such as binding a target molecule or catalyzing a specific reaction. Functional nucleic acid molecules can interact with any macromolecule, such as DNA, RNA, or carbohydrate chains. Thus, functional nucleic acids can interact with the mRNA or genomic DNA. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Representative examples of how to make and use aptamers to bind a variety of different target molecules can be found in, for example, U.S. Pat. Nos. 5,476,766 and 6,051,698.

Ribozymes are nucleic acid molecules that are capable of catalyzing a chemical reaction, either intramolecularly or intermolecularly. There are a number of different types of ribozymes that catalyze nuclease or nucleic acid polymerase type reactions which are based on ribozymes found in natural systems, such as hammerhead ribozymes, hairpin ribozymes and tetrahymena ribozymes. There are also a number of ribozymes that are not found in natural systems, but which have been engineered to catalyze specific reactions de novo (for example, but not limited to U.S. Pat. Nos. 5,807,718, and 5,910,408). Ribozymes may cleave RNA or DNA substrates. Representative examples of how to make and use ribozymes to catalyze a variety of different reactions can be found in U.S. Pat. Nos. 5,837,855; 5,877,022; 5,972,704; 5,989,906; and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. Representative examples of how to make and use triplex forming molecules to bind a variety of different target molecules can be found in U.S. Pat. Nos. 5,650,316; 5,683,874; 5,693,773; 5,834,185; 5,869,246; 5,874,566; and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. Representative examples of how to make and use EGS molecules to facilitate cleavage of a variety of different target molecules can be found in U.S. Pat. Nos. 5,168,053; 5,624,824; 5,683,873; 5,728,521; 5,869,248; and 5,877,162.

Gene expression can also be effectively silenced in a highly specific manner through RNA interference (RNAi). Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit (Ambion, Austin, Tex.).

As used herein, the term IP3R agonist refers to IP3R agonists and analogues and derivatives thereof, including, for example, natural or synthetic functional variants which have IP3R biological activity, as well as fragments of an IP3R agonist having IP3R biological activity. As further used herein, the term IP3R biological activity refers to activity that enhances or improves phosphorylation of IP3R1 at Ser⁴²¹ and/or Thr⁷⁹⁹. Commonly known IP3R agonists include, but are not limited to, the cdc2/CyB (cdk1/CyB) complex as well as other similar chemical substances, peptides or small molecules capable of combining with a the IP3R1 receptor and initiating IP3R biological activity.

IP3R1 agonists can include novel small molecules (e.g., organic compounds), and polypeptides, oligonucleotides, as well as novel natural products (preferably, in isolated form). IP3R agonists include are sigma agonists, which were originally believed to target opiate-related receptors but have been reported to interact with an ER receptor associated with 220 kDa ankyrin-B and IP3R (Hayashi and Su, Proc. Natl. Acad. Sci. USA 9:9 (2001)).

IP3R1 agonists also include antibodies. Antibodies to IP3R1 are known in the art and are described in, for example, U.S. Publication No. 2005/0119179. Antibodies to IP3R1 are also commercially available from, for example, Santa Cruz Biotechnology, Inc. (Santa Cruz, Calif.).

As used throughout, the term antibody is used herein in a broad sense and includes both polyclonal and monoclonal antibodies. Monoclonal antibodies can be made using any procedure that produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

Digestion of antibodies to produce fragments thereof, e.g., Fab fragments, can be accomplished using routine techniques known in the art. For instance, digestion can be performed using papain. Examples of papain digestion are described in WO 94/29348 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

The antibody fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term antibody or antibodies can also refer to a human antibody and/or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86 95, 1991). Human antibodies (and fragments thereof) can also be produced using phage display libraries (Hoogenboom et al., J. Mol. Biol., 227:381, 1991; Marks et al., J. Mol. Biol., 222:581, 1991). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551 255 (1993); Jakobovits et al., Nature, 362:255 258 (1993); Bruggermann et al., Year in Immunol., 7:33 (1993)). Specifically, the homozygous deletion of the antibody heavy chain joining region (J(H)) gene in these chimeric and germ line mutant mice results in complete inhibition of endogenous antibody production, and the successful transfer of the human germ line antibody gene array into such germ line mutant mice results in the production of human antibodies upon antigen challenge.

Antibody humanization techniques generally involve the use of recombinant DNA technology to manipulate the DNA sequence encoding one or more polypeptide chains of an antibody molecule. Accordingly, a humanized form of a non human antibody (or a fragment thereof) is a chimeric antibody or antibody chain that contains a portion of an antigen binding site from a non-human (donor) antibody integrated into the framework of a human (recipient) antibody. Fragments of humanized antibodies are also useful in the methods taught herein. As used throughout, antibody fragments include Fv, Fab, Fab′, or other antigen binding portion of an antibody. Methods for humanizing non human antibodies are well known in the art. For example, humanized antibodies can be generated according to the methods of Winter and co workers (Jones et al., Nature, 321:522 525 (1986), Riechmann et al., Nature, 332:323 327 (1988), Verhoeyen et al., Science, 239:1534 1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

The agents described herein are optionally administered in vivo in a pharmaceutically acceptable carrier. By pharmaceutically acceptable is meant a material that is not biologically or otherwise undesirable. Thus, the material may be administered to a subject, without causing undesirable biological effects or interacting in a deleterious manner with any of the other components of the pharmaceutical composition in which it is contained. The carrier would naturally be selected to minimize any degradation of the active ingredient and to minimize any adverse side effects in the subject.

The provided agents and compositions may be administered orally, parenterally (e.g., intravenously), by intramuscular injection, by intraperitoneal injection, transdermally, extracorporeally, topically or the like, including intranasal administration or administration by inhalant. The dosage of the agent or composition required will vary from subject to subject, depending on the species, age, weight and general condition of the subject, the severity of the airway disorder being treated, the particular active agent used, its mode of administration and the like. Thus, it is not possible to specify an exact amount for every composition. However, an appropriate amount can be determined by one of ordinary skill in the art using only routine experimentation given the teachings herein.

Suitable carriers and their formulations are described in Remington: The Science and Practice of Pharmacy (21 st ed.) eds. A. R. Gennaro et al., University of the Sciences in Philadelphia 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8.5, and more preferably from about 7.8 to about 8.2. Further carriers include sustained release preparations such as semipermeable matrices of solid hydrophobic polymers containing the antibody, which matrices are in the form of shaped articles, e.g., films, liposomes or microparticles. Certain carriers may be more preferable depending upon, for instance, the route of administration and concentration of composition being administered. Pharmaceutical carriers are known to those skilled in the art. These most typically would be standard carriers for administration of drugs to humans, including solutions such as sterile water, saline, and buffered solutions at physiological pH. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The terms effective amount and effective dosage are used interchangeably. The term effective amount is defined as any amount necessary to produce a desired physiologic response. Effective amounts and schedules for administering the compositions may be determined empirically. The dosage ranges for the administration of the compositions are those large enough to produce the desired effect in which the symptoms or disorder are affected. The dosage should not be so large as to cause substantial adverse side effects, such as unwanted cross-reactions, anaphylactic reactions, and the like.

The provided compositions can be administered in combination with one or more other therapeutic or prophylactic regimens. As used throughout, a therapeutic agent is a compound or composition effective in ameliorating a pathological condition. Illustrative examples of therapeutic agents include, but are not limited to, an anti-cancer compound, anti-diabetic agents, anti-inflammatory agents, anti-viral agents, anti-retroviral agents, anti-opportunistic agents, antibiotics, immunosuppressive agents, immunoglobulins, and antimalarial agents.

For example, the provided agents, agonists and compositions can be administered in combination with an anti-diabetic agent. Anti-diabetics include, but are not limited to, insulin and analogues and derivatives thereof, camitine, taurine, sulfonylureas such as glibenclamide (glyburide); biguanides such as metformin and phenformin; thiazolidinediones (TZDs) such as rosiglitazone, pioglitazone, and troglitazone; α-glucosidase inhibitors such as acarbose and miglitol; meglitinides such as nateglinide, repaglinide, and their analogs; incretin mimetics and insulin secretagogues including, glucagon-like peptide (GLP) analogs, exenatide, liraglutide, gastric inhibitory peptide (GIP) analogs; dipeptidyl peptidase-4 (DPP-4) inhibitors such as sitagliptin and amylin agonist analogs such as pramlintide.

The provided agents, agonists and compositions can also be administered in combination with an anti-obesity agent. Anti-obesity agents or drugs include, but are not limited to, fluoxetine, orlistat, and sibutramine.

Any of the aforementioned treatments can be used in any combination with the compositions described herein. Combinations can be administered as desired by those of skill in the art. Combinations may be administered either concomitantly (e.g., as an admixture), separately but simultaneously (e.g., via separate intravenous lines into the same subject), or sequentially (e.g., one, of the compounds or agents is given first followed by the second). Thus, the term combination is used to refer to either concomitant, simultaneous, or sequential administration of two or more agents.

As used throughout, by a subject is meant an individual. Thus, the subject can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, a human.

As used herein, references to decreasing, reducing, or inhibiting include a change of 10, 20, 30, 40, 50, 60, 70, 80, 90 percent or greater as compared to a control level. Such terms can include but do not necessarily include complete elimination.

As used herein the terms treatment, treat or treating refers to a method of reducing the effects of a disease or condition or symptom of the disease or condition. Thus in the disclosed method treatment can refer to a 10%, 20%, 30%, 40%, 50%, 60%, 70%, 80%, 90% or 100% reduction in the severity of an established disease or condition or symptom of the disease or condition. For example, a method for treating a disease is considered to be a treatment if there is a 10% reduction in one or more symptoms of the disease in a subject as compared to control. Thus the reduction can be a 10, 20, 30, 40, 50, 60, 70, 80, 90, 100% or any percent reduction in between 10 and 100 as compared to native or control levels. It is understood that treatment does not necessarily refer to a cure or complete ablation of the disease, condition or symptoms of the disease or condition.

As used herein, the terms prevent, preventing and prevention of a disease or disorder refers to an action including, for example, administration of a therapeutic agent, that occurs before a subject begins to suffer from one or more symptoms of the disease or disorder, which inhibits or delays onset of the severity of one or more symptoms of the disease or disorder.

With the knowledge of the sequence and or gene structure of the cDNA or genomic DNA encoding GRP78 and IP3R1 and regulatory sequences regulating expression thereof, it is possible to generate transgenic animals with genotype of GRP78+/− or IP3R1+/−. There are a variety of sequences related to, for example, Grp78 and IP3R1 that are disclosed on Genbank, at www.pubmed.gov, and these sequences and others are herein incorporated by reference in their entireties as well as for individual subsequences contained therein. For example, the amino acid sequence for the mouse and human IP3R1 can be found at Genbank Accession Nos. NP_{—}034715 and Q14643, respectively. The nucleic acid sequence for the mouse and human IP3R1, for example, can be found at Genbank Accession Nos. NM_{—}010585 and L38019.2, respectively.

Thus, provided are non-human animals containing a disruption in an endogenous GRP78 gene such that the GRP78 gene is non-functional or does not express a functional GRP78 protein are provided. Also provided are non-human animals comprising a disruption in an endogenous IP3R1 gene such that the IP3R1 gene is non-functional or does not express a functional IP3R1 protein, wherein the disruption is an insertion of a transgene into the endogenous IP3R1 gene. The disruption can be, for example, an insertion, missense, frameshift, or deletion mutation. The disruption can also alter a promoter, enhancer, or splice site. The disruption can be insertion of a transgene. The transgene optionally encodes a selectable marker, such as, for example a LacZ reporter gene operably linked to a GRP78 promoter. The provided non-human animals are heterozygous for GRP78 or IP3R1. As used herein, the term heterozygous means that the animal has a disruption in one allele (i.e., endogenous gene) while the second allele is unaffected (i.e., does not contain a disruption).

The animals heterozygous for IP3R1 exhibit one or more phenotypes associated with a disorder associated with weight gain, and are useful as models of such disorders including, for example, obesity and type II diabetes. Thus, the animals heterozygous for IP3R1 are useful for drug screening, e.g., to identify agents that treat a disorder associated with weight gain, that reduce the risk that a subject will develop a disorder associated with weight gain, and the like. The transgenic animals heterozygous for IP3R1 or GRP78 are also useful in research applications, for studying, e.g., the effects of diet and other factors on disorders associated with weight gain.

The provided transgenic animals are other than human, and are typically non-human mammals, including, but not limited to pigs, goats, sheep, cows, horses, rodents (such as mice), and lagomorphs (e.g., rabbits). Thus, the non-human animal can be a mouse.

Any method of making transgenic animals can be used as described in the Examples below and as described in the art, for example, in Transgenic Animal Generation and Use, L. M. Houdebine, ed. Harwood Academic Press, 1997; Transgenesis Techniques: Principles and Protocols, D. Murphy and D. A. Carter, eds. Humana Press, 1993; Transgenic Animal Technology: A Laboratory Handbook, C. A. Pinkert, ed. Academic Press, 1994; Transgenic Animals, F. Grosveld and G Kollias, eds. Academic Press, 1992; and Embryonal Stem Cells: Introducing Planned Changes into the Animal Germline, M. L. Hooper, ed. Gordon & Breach Science Pub., 1993; U.S. Pat. No. 6,344,596; U.S. Pat. No. 6,271,436; U.S. Pat. No. 6,218,596; and U.S. Pat. No. 6,204,431; Maga and Murray, (1995) Bio/Technol. 13:1452-1457; Ebert et al., (1991) Bio/Technol. 9:835-838; Velander et al., (1992) Proc. Natl. Acad. Sci. USA 89:12003-12007; Wright et al., (1991) Bio/Technol. 9:830-834.

Transgenic animals also can be generated using methods of nuclear transfer or cloning using embryonic or adult cell lines as described in, for example, Campbell et al., (1996) Nature 380:64-66; and Wilmut et al., (1997) Nature 385: 810-813. Cytoplasmic injection of DNA can be used, as described in U.S. Pat. No. 5,523,222. Transgenic animals also include somatic transgenic animals, e.g., transgenic animals that include a transgene in somatic cells (and not in germ line cells). Methods of somatic cell transformation are described in the art as described in, for example, Furth et al. (1995) Mol. Biotechnol. 4:121-127.

As described above, the animals heterozygous for IP3R1 are useful for drug screening, e.g., to identify agents that treat a disorder associated with weight gain, that reduce the risk that a subject will develop a disorder associated with weight gain, and the like. Thus, provided are methods of screening for an agent that prevents or treats a disorder associated with weight gain, comprising administering an agent to be tested to a non-human animal heterozygous for IP3R1 and detecting fasting insulin level or detecting glucose intolerance in the non-human animal. An increase in fasting insulin levels indicates the agent prevents or treats a disorder associated with weight gain. A decrease in glucose intolerance indicates the agent is suitable for preventing or treating a disorder associated with weight gain. The disorder can be, for example, obesity or type II diabetes. The obesity can be diet-induced obesity.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a genetic manipulation is disclosed and discussed and a number of modifications that can be made to a number of genes or a number of sites including the site and gene are discussed, each and every combination and permutation of the gene and site and the modifications that are possible are specifically contemplated unless specifically indicated to the contrary. Thus, if a class of molecules A, B, and C are disclosed as well as a class of molecules D, E, and F and an example of a combination molecule, A-D, is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination).

Throughout this application, various publications are referenced. The disclosures of these publications in their entireties are hereby incorporated by reference into this application.

A number of embodiments have been described. Nevertheless, it will be understood that various modifications may be made. Furthermore, when one characteristic or step is described it can be combined with any other characteristic or step herein even if the combination is not explicitly stated. Accordingly, other embodiments are within the scope of the claims.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention except as and to the extent that they are included in the accompanying claims. Efforts have been made to ensure accuracy with respect to numbers (e.g., amounts, temperature, etc.), but some errors and deviations should be accounted for.

### EXAMPLES

### Example 1

### GRP78 Heterozygous Mice are Resistant to Obesity and Diabetes

To determine directly the function of GRP78 in vivo, knock-out mouse models of GRP78 were created. While the homozygous knockout mice are embryonic lethal, the heterozygous Grp78+/− mice which express 50% level of the wild-type (WT) level of GRP78 are viable (Luo et al., 2006). The Grp78+/− mice and its WT siblings, when fed on regular diet (RD), showed no obvious phenotype in body weight and diabetes. When the Grp78+/− mice were backcrossed to C57BL/6, they were more resistant to high fat diet (HFD)-induced obesity. In contrast HFD-induced obesity was apparent in the WT siblings following 4 weeks of HFD (C57BL/6 mice are known to develop obesity and hyperglycemia when on a HFD). The resistance to obesity is not due to a lack of food intake since the Grp78+/− mice showed similar food consumption as the WT siblings. The resistance to obesity is also not due to inability of the Grp78+/− mice to absorb fat since stool smear tests did not show staining of unabsorbed fat droplets in the mutant mice, in contrast to the intense staining using adipose tissue extract as positive control. It was further determined that the Grp78+/− mice fed a HFD were more resistant to the development of hyperglycemia, which was observed in the WT mice by 13 week.

In addition, Grp78+/− mice fed on high fat diet did not exhibit β-cell hyperplasia, which was observed in the WT sibling. Insulin staining of the islets further revealed that even under regular diet (RD) conditions there was less homogeneity in the staining pattern of the mutant mice as compared to the WT, indicating that there are changes in the β-cell integrity of the heterozygous mice. Under HFD conditions, insulin staining was intense in the expanded islets of the WT mice. For the GRP78+/− mice, the staining intensity was the same as under RD but the pattern was more diffused. Consistent with the immunhistochemical staining results, the fasting insulin level of Grp78+/− mice fed on HFD is 40% of WT. The lower but sufficient level of insulin produced by the Grp78+/− mice can lead to less fat storage and/or less insulin resistance, thereby the mice remain relatively lean despite continuous regimen of HFD for 20 weeks.

The lack of obesity also leads to resistance to the subsequent development of diet-induced diabetes in the Grp78+/− mice. Grp78+/− mice showed less insulin resistance than WT siblings fed on HFD, while no difference was observed under regular diet conditions. In addition, faster clearance of blood glucose was observed in GRP78+/− mice. These data indicate that reduction of GRP78 levels suppresses HFD-induced obesity and diabetes.

### Example 2

### D2D as a Mouse Model for Type II Diabetes and Obesity

To understand regulation of Grp78 in vivo, multiple transgenic mouse lines with various modified versions of the Grp78 promoter driving the expression of the bacterial LacZ gene encoding for p-galactosidase were created (Mao et al., 2004). As described herein, it was discovered that fed on a regular diet (RD), one line (D2D) became dramatically obese with age. After inter-sibling breeding through three to four generations, the age-dependent onset of obesity was still observed. The level of obesity observed is clinically relevant as it mimics the age-dependent onset of obesity in the human population. MRI scanning revealed substantial accumulation of abdominal fat in the D2D mice. Correlating with the obesity, the D2D mice developed progressing glucose intolerance and hyperglycemia. In 12 week old D2D mice glucose intolerance is apparent although hyperglycemia was detected only after 24 week of age.

The D2D pancreatic sections showed normal range of β-cell mass, proliferation and apoptosis, as compared to sex- and age-matched normal littermate control mice. Despite an elevated fasting glucose level, they are not hyperinsulinemic. Rather, the fasting insulin level for the D2D mice is about 70% of the WT level. These results indicate that the hyperglycemia observed in the D2D line are due to β-cell dysfunction such as a defect in insulin secretion rather than deficit in β-cell mass.

Using an established method of inverse PCR, the integration site of the transgene in the D2D line was determined. The transgene was inserted into mouse chromosome 6, disrupting the Ca2+ receptor gene IP3R1. The insertion event in the mouse genome, as well as the orientation of the transgene insertion into the mouse genome, were confirmed by PCR using mouse genomic DNA as template and PCR primers external to the transgene paired with primers located inside the transgene. By Western blot, it was determined that the level of the ˜225 kDa IP3R1 protein was reduced by half in D2D adult tissues as expected for disruption of one gene allele. Further analysis revealed that the transgene insertion event also resulted in the deletion of 11 identified genes on chromosome 6 directly adjacent to the IP3R1 gene.

IP3R1 (inositol 1,4,5-triphosphate receptor 1) is an ER transmembrane protein and is predominant in mouse brain and pancreatic islets as the major regulator of Ca2+ efflux from the ER (Lee and Laychock, 2001). The IP3R1 protein binds the intracellular second messenger IP3, which is generated by phsopholipase C-mediated hydrolysis of phosphatidylinositol 4,5-bisphosphate (Berridge, 1993). Ligand binding triggers the efflux of calcium from intracellular stores, suggesting the IP3R1 is both a receptor of IP3 and a Ca2+ channel. Since Ca2+ signaling may be critical in glucose stimulated secretion of insulin (Srivastava et al., 1999), disruption of IP3R1 could result in impairment of Ca2+ efflux from the ER and subsequently of insulin secretion. In fact, the fasting insulin level for the D2D mice is lower than the WT level. To test directly whether the D2D phenotype is at least in part due to IP3R1 disruption, a mouse strain was obtained from the Jackson Laboratory (Bar Harbor, Me.) referred to as the opisthotonoas (Opt) mouse, which carries a mutation in one IP3R1 allele. The opt mutation is a single gene mutation which arose spontaneously in a C57BL/Ks-db2J colony. The mutant mouse was first identified based on its ataxic and convulsive phenotype (Street et al., 1997). Genetic techniques were used to localize opt to distal chromosome 6, and subsequently molecular genetic studies identified that the opt mutation is due disruption of the IP3R1 gene. A targeted disruption of the IP3R1 gene was shown to exhibit phenotypes similar to that of opt (Matsumoto et al., 1996). Similar to the D2D mice, 10-week old heterozygous opt mice exhibit glucose intolerance but not the insulin resistance. This indicates that the opt mice have normal insulin intake into the muscle or liver, but a defect in insulin secretion. It was also determined whether opt mice fed on a HFD exacerbated the development of hyperglycemia and obesity. Heterozygous opt mice fed on a HFD exhibit hyperglycemia and increased body mass more rapidly than WT siblings.

### REFERENCES

Allayee H, Andalibi A, Mehrabian M. Using inbred mouse strains to identify genes for complex diseases. Front Biosci 11:1216-1226, 2006.
Berridge M J. Inositol triphosphate and calcium signalling. Nature 361:315-325, 1993.
Bertolotti A, Zhang Y. Hendershot L M, Harding H P, Ron D. Dynamic interaction of BiP and ER stress transducers in the unfolded-protein response. Nat Cell Biol. 2:326-332, 2000.
Butler A E, Janson J, Soeller W C, Butler P C. Increased beta-cell apoptosis prevents adaptive increase in beta-cell mass in mouse model of type 2 diabetes: evidence for role of islet amyloid formation rather than direct action of amyloid. Diabetes 52:2304-2314, 2003.
Chua S C, Jr., Chung W K, Wu-Peng X S, Zhang Y, Liu S M, Tartaglia L, Leibel R L. Phenotypes of mouse diabetes and rat fatty due to mutations in the OB (leptin) receptor. Science 271:994-996, 1996.
Dong D, Dubeau L, Bading J, Nguyen K, Luna M, Yu H, Gazit-Bornstein G, Gordon E M, Gomer C, Hall F L, Gambhir S, Lee A S. Spontaneous and controllable activation of suicide gene expression driven by the stress-inducible grp78 promoter resulting in eradication of sizable human tumors. Hum Gene Ther 15:553-561, 2004.
Duhl D M, Vrieling H, Miller K A, Wolff G L, Barsh G S. Neomorphic agouti mutations in obese yellow mice. Nat Genet 8:59-65, 1994.
Ellgaard L, Molinari M, Helenius A. Setting the standards: quality control in the secretory pathway. Science 286:1882-1888, 1999.
Fu Y, Lee A S. Glucose regulated proteins in cancer progression, drug resistance and immunotherapy. Cancer Biol. Ther 5:741-744, 2006.
Gazit G, Hung G, Chen X, Anderson W F, Lee A S. Use of the glucose-starvation inducible grp78 promoter in suicide gene therapy of murine fibrosarcoma. Cancer Res 59:3100-3106, 1999.
Haas I G, Wabl M. Immunoglobulin heavy chain binding protein. Nature 306:387-389, 1983.
Harding H P, Zeng H, Zhang Y, Jungries R, Chung P, Plesken H, Sabatini D D, Ron D. Diabetes mellitus and exocrine pancreatic dysfunction in perk−/− mice reveals a role for translational control in secretory cell survival. Mol Cell 7:1153-1163, 2001.
Hong M, Luo S, Baumeister P, Huang J M, Gogia R K, Li M, Lee A S. Underglycosylation of ATF6 as a novel sensing mechanism for activation of the unfolded protein response. J. Biol. Chem 279:11354-11363, 2004.
Jamora C, Dennert G, Lee A S. Inhibition of tumor progression by suppression of stress protein GRP78/BiP induction in fibrosarcoma B/C10ME. Proc Natl. Acad Sci USA 93:7690-7694, 1996.
Kaufman R J. Stress signaling from the lumen of the endoplasmic reticulum: coordination of gene transcriptional and translational controls. Genes Dev 15 13:1211-1233, 1999.
Kaufman R J, Scheuner D, Schroder M, Shen X, Lee K, Liu C Y, Arnold S M. The unfolded protein response in nutrient sensing and differentiation. Nat Rev Mol Cell Biol. 3:411-421, 2002.
Kayo T, Koizumi A. Mapping of murine diabetogenic gene mody on chromosome 7 at D7Mit258 and its involvement in pancreatic islet and beta cell development during the perinatal period. J Clin Invest 101:2112-2118, 1998.
Lee A S. Coordinated regulation of a set of genes by glucose and calcium ionophores in mammalian cells. Trends Biochem Sci 12:20-23, 1987.
Lee A S. The glucose-regulated proteins: stress induction and clinical applications. Trends Biochem Sci 26:504-510, 2001.
Lee B, Laychock S G, Inositol 1,4,5-trisphosphate receptor isoform expression in mouse pancreatic islets: effects of carbachol. Biochem Pharmacol 61:327-336, 2001.
Li J, Lee A S. Stress induction of GRP78/BiP and its role in cancer. Curr Mol Med 6:45-54, 2006.
Lievremont J P, Rizzuto R, Hendershot L, Meldolesi J. BiP, a major chaperone protein of the endoplasmic reticulum lumen, plays a direct and important role in the storage of the rapidly exchanging pool of Ca2+. J. Biol. Chem 272:30873-30879, 1997.
Luo S, Mao C, Lee B, Lee A S. GRP78IBiP is required for cell proliferation and protecting the inner cell mass from apoptosis during early mouse embryonic development. Mol Cell Biol. 26:5688-5697, 2006.
Mao C, Dong D, Little E, Luo S, Lee A S. Transgenic mouse models for monitoring endoplasmic reticulum stress in vivo. Nat Med 10:1013-1014, 2004.
Mathis D, Vence L, Benoist C. beta-Cell death during progression to diabetes. Nature 414:792-798, 2001.
Matsumoto M, Nakagawa T, Inoue T, Nagata E, Tanaka K, Takano H, Minowa O, Kuno J, Sakakibara S, Yamada M, Yoneshima H, Miyawaki A, Fukuuchi Y, Furuichi T, Okano H, Mikoshiba K, Noda T. Ataxia and epileptic seizures in mice lacking type 1 inositol 1,4,5-trisphosphate receptor. Nature 379:168-171, 1996.
Munro S, Pelham H R. An Hsp70-like protein in the ER: identity with the 78 kd glucose-regulated protein and immunoglobulin heavy chain binding protein. Cell 46:291-300, 1986.
Pelleymounter M A, Cullen M J, Baker M B, Hecht R, Winters D, Boone T, Collins F. Effects of the obese gene product on body weight regulation in ob/ob mice. Science 269:540-543, 1995.
Rao R V, Castro-Obregon S, Frankowski H, Schuler M, Stoka V, del Rio G, Bredesen D E, Ellerby H M. Coupling endoplasmic reticulum stress to the cell death program. An Apaf-1-independent intrinsic pathway. J. Biol. Chem 277:21836-21842, 2002.
Reddy R K, Mao C, Baumeister P, Austin R C, Kaufman R J, Lee A S. Endoplasmic reticulum chaperone protein GRP78 protects cells from apoptosis induced by topoisomerase inhibitors: role of ATP binding site in suppression of caspase-7 activation. J. Biol. Chem 278:20915-20924, 2003.
Rhodes C J, White M F. Molecular insights into insulin action and secretion. Eur J Clin Invest 32 SuppI3:3-13, 2002.
Rutkowski D T, Kaufman R J. A trip to the ER: coping with stress. Trends Cell Biol. 14:20-28, 2004.
Shen J, Chen X, Hendershot L, Prywes R. ER stress regulation of ATF6 localization by dissociation of BiP/GRP78 binding and unmasking of golgi localization signals. Dev Cell 3:99-111, 2002.
Shiu R P, Pouyssegur J, Pastan 1. Glucose depletion accounts for the induction of two transformation-sensitive membrane proteins in Rous Sarcoma virus-transformed chick embryo fibroblasts. Proc Natl Acad Sci USA 74:3840-3844, 1977.
Sommer T, Jarosch E. BiP binding keeps ATF6 at bay. Dev Cell 3:1-2, 2002.
Srivastava M, Atwater I, Glasman M, Leighton X, Goping G, Caohuy H, Miller Q Pichel J, Westphal H, Mears D, Rojas E, Pollard H B. Defects in inositol 1,4,5-trisphosphate receptor expression, Ca(2+) signaling, and insulin secretion in the anx7(+/−) knockout mouse. Proc Natl Acad Sci USA 96:13783-13788, 1999.
Street V A, Bosma M M, Demas V P, Regan M R, Lin D D, Robinson L C, Agnew W S, Tempel B L. The type 1 inositol 1,4,5-trisphosphate receptor gene is altered in the opisthotonos mouse. J Neurosci 17:635-645, 1997.
Wellen K E, Hotamisligil G S. Inflammation, stress, and diabetes. J Clin Invest 115:1111-1119, 2005.
Yang X J, Kow L M, Funabashi T, Mobbs C V. Hypothalamic glucose sensor: similarities to and differences from pancreatic beta-cell mechanisms. Diabetes 48:1763-1772, 1999.
Ye J, Rawson R B, Komuro R, Chen X, Dave U P, Prywes R, Brown M S, Goldstein J L. ER stress induces cleavage of membrane-bound ATF6 by the same proteases that process SREBPs. Mol Cell 6:1355-1364, 2000.

What is claimed is:

1. A method of increasing resistance to a disorder associated with weight gain in a subject comprising:
a) selecting a subject at risk for developing a disorder associated with weight gain; and
b) administering to the subject one or more agents that inhibit expression or activity of GRP78.

2. A method of treating a disorder associated with weight gain in a subject comprising:
a) selecting a subject that has a disorder associated with weight gain; and
b) administering to the subject one or more agents that inhibit expression or activity of GRP78.

3. The method of claim 1, wherein expression of GRP78 protein is inhibited.

4. The method of claim 1, wherein the activity of GRP78 is inhibited.

5. The method of claim 4, wherein the agent that inhibits activity of GRP78 is a GRP78 antagonist.

6. The method of claim 5, wherein the GRP78 antagonist is (−)-epigallocatechin gallate or genistein.

7. The method of claim 5, wherein the GRP78 antagonist is an antibody.

8. The method of any claim 1, further comprising administering IP₃ to the subject.

9. The method of claim 1, wherein the disorder is obesity.

10. The method of claims 1, wherein the disorder is type II diabetes.

11. A method of increasing resistance to a disorder associated with weight gain in a subject comprising:
a) selecting a subject at risk for developing a disorder associated with weight gain; and
b) administering to the subject one or more IP3R1 agonists.

12. A method of treating a disorder associated with weight gain in a subject comprising:
a) selecting a subject that has a disorder associated with weight gain; and
b) administering to the subject one or more IP3R1 agonists.

13. The method of claim 11, wherein the IP3R1 agonist is selected from the group consisting of polypeptides, small molecules and antibodies.

14. The method of claim 11, wherein the IP3R1 agonist is an antibody.

15. The method of claim 11, wherein the IP3R1 agonist is IP₃.

16. The method of claim 11, wherein the disorder is obesity.

17. The method of claim 11, wherein the disorder is type II diabetes.

18. A non-human animal comprising a disruption in an endogenous GRP78 gene such that the GRP78 gene is non-functional or does not express a functional GRP78 protein.

19. The non-human animal of claim 18, wherein the animal is heterozygous for the GRP78 disruption.

20. A non-human animal comprising a disruption in an endogenous IP3R1 gene such that the IP3R1 gene is non-functional or does not express a functional IP3R1 protein, wherein the disruption is an insertion of a transgene into the endogenous IP3R1 gene.

21. The non-human animal of claim 20, wherein the animal is heterozygous for the IP3R1 disruption.

22. The non-human animal of claim 20, wherein the transgene encodes a LacZ reporter gene operably linked to a GRP78 promoter.

23. A method of screening for an agent that prevents or treats a disorder associated with weight gain, comprising:
a) administering an agent to be tested to the non-human animal of claim 34; and
b) detecting fasting insulin level in the non-human animal, wherein an increase in fasting insulin levels indicates the agent prevents or treats a disorder associated with weight gain.

24. The method of claim 23, wherein the disorder is obesity.

25. The method of claim 23, wherein the disorder is type II diabetes.
